(19) United States
(12) Patent Application Publication
(10) Pub. No.: US 2015/0094291 A1
(43) Pub. Date: Apr. 2, 2015

(54) Method and Substance for Improving the Emotional Development of an Infant

(71) Applicant: Opendot Research Co Limited, Kent (GB)

(72) Inventor: Derek John GOLDSMITH, Edenbridge (GB)

(73) Assignee: Opendot Research Co Limited, Kent (GB)

(21) Appl. No.: 14/044,379

(22) Filed: Oct. 2, 2013

(51) Int. Cl. A61K 31/575; A61K 31/565; A61K 31/566

(52) U.S. Cl. CPC A61K 31/575; A61K 31/566; A61K 31/565
USPC 514/179; 514/178; 514/182; 514/177; 552/625; 552/646; 552/553

(57) ABSTRACT

The invention provides a method for improving the emotional development of an infant by performing controlled exposure of the infant aged less than six months old to human fear pheromone, preferably with visual fear stimuli, so that the natural synaptic connections in the brain associated with normal fear mechanisms are stimulated and exercised. This can reduce the occurrence of mental illness later in life, in particular reduce the occurrence of schizophrenia. Advantageously the exposure to fear pheromone and visual fear stimuli is terminated and followed by comforting stimuli such as feeding, physical contact, or comforting or happy visual or audible stimuli such as laughter.

### FIELD OF THE INVENTION

The present invention relates to improving the development of the mental state of an infant, for example to reduce the susceptibility of the infant to developing psychological disorders such as schizophrenia.

PRIOR ART

In my U.S. Pat. No. 6,058,939 it is proposed that an infant be caused to have its sleep pattern controlled in order to ensure that as much as possible of the sleep experienced by the infant is rapid eye motion (REM) sleep. The method there proposed involved waking the infant when it had not entered REM sleep and then soothing the wakened infant to allow it to fall asleep once more, in a relaxed state more likely to lead to REM sleep. This method was based on the belief that in REM sleep the emotional development of the infant is better than in non-REM sleep when the infant is in what I there termed a “frozen” state, reminiscent of a particularly helpful reflex in the rest of the animal kingdom and in particular in primitive man, where the infant would remain silent and motionless in order to reduce the likelihood of interference by a predator or other threat.

There is now increased interest in exploring how development in the early months of life affects mental illness later in life.

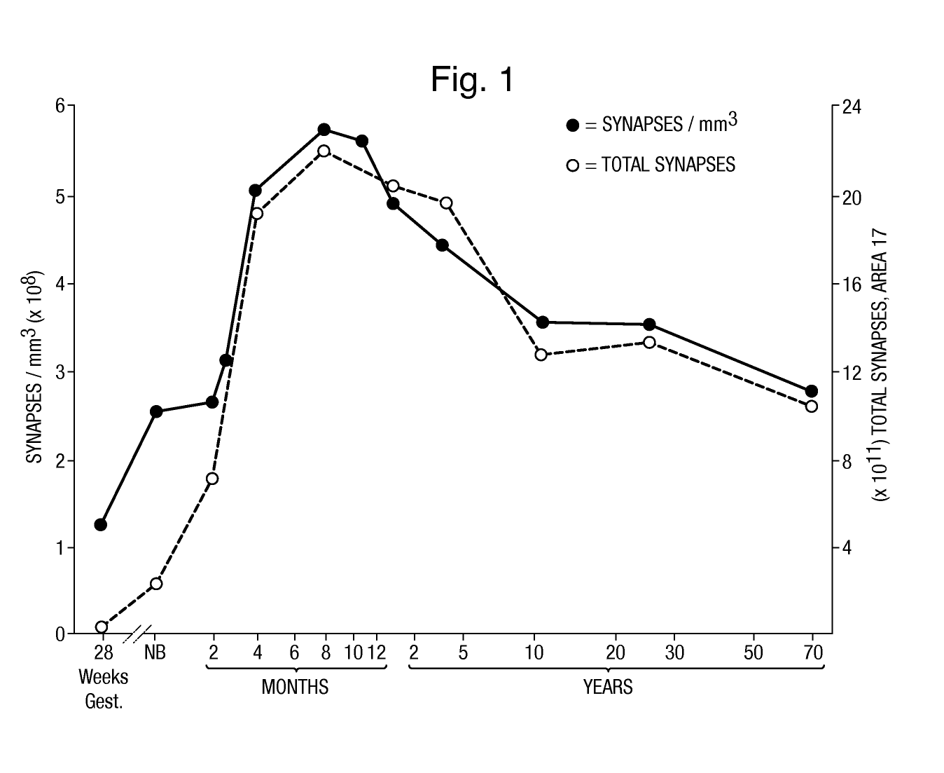

It has recently been discovered that there are no cases of blind-from-birth schizophrenics suggesting a role of visual stimuli in schizophrenia. Furthermore it has also emerged recently from twin separation studies that schizophrenia is associated with some form of precipitating event which occurs between 3 and 6 months of age. As illustrated by FIG. 1 of the accompanying drawings the density of synaptic connections in the visual cortex of the human brain increases rapidly in the first 8 months of life.

### OBJECTS OF THE INVENTION

Accordingly, it is a first object of the present invention to provide a method for improving the emotional development of an infant comprising performing controlled exposure of the infant to a human fear pheromone under medical supervision while the infant is less than six months old.

Modern humans have inherited a fear system that is far too powerful for modern civilized living. In fact infants are extremely unlikely to encounter truly fearful situations in the early years of life. This can lead to the fear pathways in the infant's brain being incorrectly stimulated by other strong emotions such as anger. It is a characteristic of schizophrenia that the sufferer is unable to distinguish between different strong or negative emotions in other individuals, for example between fear, anger, disgust and sadness. The present invention uses controlled exposure to the human fear pheromone to stimulate correct synaptic connection in the infant brain so that the fear pathway is not incorrectly triggered by other strong emotions such as anger.

Pheromones are well-known as secreted or excreted chemical factors which trigger a response in other humans. They act as chemical messengers transported outside the body—through the air—are sensed by the olefactory system of another human and result in a direct effect on hormone and/or behaviour in the other human. The fear or alarm pheromone (the two are the same) is produced in response to stress in a threatening situation. It appears in sweat and acts via the olefactory system of others nearby.

In one embodiment the controlled exposure to the fear pheromone is performed while the infant is from three to six months old, preferably while simultaneously providing a visual fear stimulus to the infant. Infants can see sufficiently well from 3 months to respond to the visual stimulus. The visual fear stimulus can be a fearful human expression, e.g. from the mother, health professional, or in a picture.

The controlled exposure can performed while simultaneously inducing a frozen psychological state, e.g. by the visual stimulus mentioned above. The inducing of the natural frozen state enhances the natural development of fear pathways in the brain.

In an alternative embodiment the controlled exposure is performed while the infant is less than three months old.

Preferably upon terminating the controlled exposure to the fear pheromone the infant is comforted. This may be by at least one of feeding, preferably breast feeding (which places the infant in close proximity to the underarm area which is an important source of pheromones), soothing physical contact, soothing audible stimulation, soothing or reassuring visual stimulation such as by human facial expression. These may be achieved by exposing the infant to laughter, the mother's laughter being particularly preferred.

The human fear pheromone may be a natural or synthetic pheromone optionally comprising cortisol.

The human fear pheromone may be a compound of Formula (1):

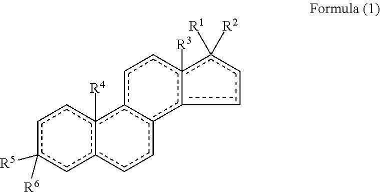

wherein:

R¹ and R² independently represent a hydrogen atom or a hydroxyl group, or R¹ and R² together represent an oxo group;

R³ represents a hydrogen atom or a methyl group, or is absent if the carbon atom to which it is attached is present in a carbon-carbon double bond

R⁵ and R⁶ independently represent a hydrogen atom or a hydroxyl group, or R⁵ and R⁶ together represent an oxo group;

R⁴ represents a hydrogen atom or a methyl group, or is absent if the carbon atom to which it is attached participates in a carbon-carbon double bond; and

[symbol] represents a carbon-carbon single bond or a carbon-carbon double bond, provided that no carbon atom participates in more than four bonds.

As used herein, the term “the carbon atom to which it is attached participates in a carbon-carbon double bond” designates a carbon atom bonded to R³ or R⁴ which is sp² hybridised.

Typically, the compound of formula (1) contains two, three or four carbon-carbon double bonds.

Typically, when one of R¹ and R² represents a hydroxyl group, the other represents a hydrogen atom. Typically, when one of R⁵ and R⁶ represents a hydroxyl group, the other represents a hydrogen atom.

In one embodiment the human fear pheromone is one of compounds 1 to 7 below.

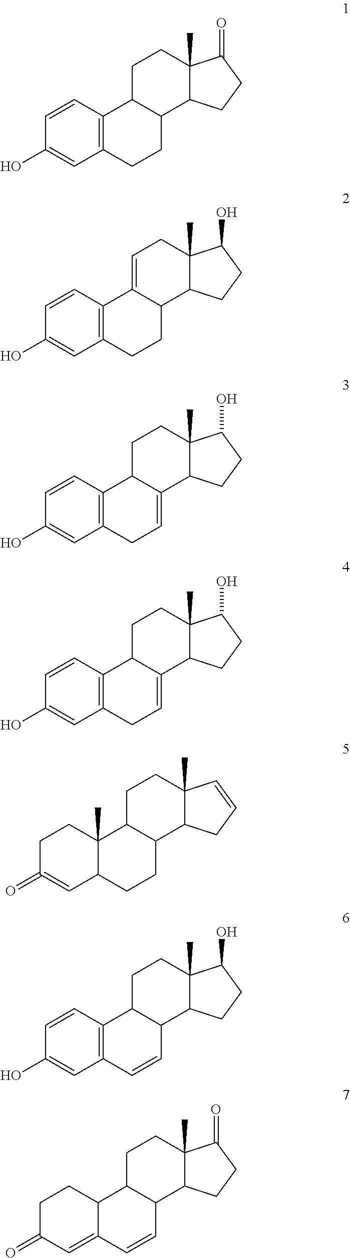

The exposure may be by an olfactory stimulus to the infant. For example the pheromone may be applied to a human holding the infant, e.g. the mother, or by a spray or vapour release in the vicinity of the infant.

The human fear pheromone may be applied in isolated form, or as part of a fear-inducing composition, e.g. a composition comprising two or more of compounds 1 to 7 above. The fear-inducing composition may be sweat obtainable from a human subject in a state of fear. For example, a human subject, typically an adult male, may be subjected to a fear-inducing stimulus or stimuli in order to induce the state of fear. Sweat may be collected from the subject in the state of fear by any suitable means, e.g. using absorbent pads placed in contact with the subject's skin. The fear inducing stimulus or stimuli may be, for example, a horror film, an animal of which the subject has a phobia (e.g. a snake, tarantula or spider), a situation perceived by the subject as potentially perilous (e.g. bungee jumping or skydiving), or a combination thereof.

The controlled exposure may be repeated monthly or weekly.

### DETAILED DESCRIPTION OF THE INVENTION

Modern humans have inherited a fear system that is far too powerful for modern civilized living. Infants can be “frozen” by seeing fear on a parent's face, or by otherwise seeing a similar facial expression. Fear is also transmitted by a mother in the form of pheromones, and this can be detected by the infant from birth. Early humans experienced fear on a daily basis. After the danger had passed the group would experience spontaneous and catching laughter that dispelled the fear-signalling pherormones and tensions of fear.

However modern life can leave this normal fear mechanism undeveloped. This has serious implications for the mental health of the child. Because the natural transmission and stimulation role of emotions does not develop properly, so the important communication process does not occur.

Not until recently have the leading world experts begun to look at how important emotions are to our development early in life. Previously emotions have been thought of only as feelings, with little attention being given to how or why they arise. Yet emotions can be expressed on the adult face within milliseconds of their cause i.e. in a sudden road accident situation. Thus the face is acting as an extension of the brain.

Since Darwin's time scientists have sought to understand why and what is the relevance of facial expressions to other humans. This is important in children relating to the new world that they are discovering, and it is important for children in the early years to read their parents' emotions correctly when they are so rapidly expressed directly in the context of the situations that give rise to them. Children typically do this out of the corner of their eye!

It is also known that young infants, 3 to 6 months, can read the mother's face and, when surprised or startled, an infant focuses with eyes wide open on that face expecting to see fear.

Most importantly it is known that much mental illness has its roots in the incorrect interpretation of other people's emotions, particularly parental emotions.

Generally, it is felt that the child or adult who is better able to read emotion is a more healthy one, and the method of the present invention proposes to improve that ability by stimulating correct development of the fear mechanisms in the infant brain using fear pheromones.

Thus the invention lies in stimulating synaptic cell connectivity in a way which mimics the manner that human evolution intended it to be connected by stimulation with a fear pheromone. This will reduce the occurrence of mental illness that develops later in life because the fear mechanisms in the brain will not be triggered incorrectly by different emotions such as anger, disgust and sadness as these will not occur with the generation of fear pheromone. In essence the fear mechanism in the brain is being conditioned to only activate in the presence of the fear pheromone.

Infants can smell pheromones from birth, but can only recognise visual stimuli from about 3-6 months. Thus two embodiments of the invention are envisaged.

In a first embodiment an infant of 0-3 months in age is exposed under medical supervision to a natural or synthetic fear pheromone, for example by spraying the pheromone in the vicinity of the infant or applying it to the body or clothing of a person holding the infant. This exposure will exercise the normal fear pathways in the brain. After exposure, for example for a few minutes, the pheromone is removed and the infant then comforted, for example by feeding or soothing visual or oral stimuli such as the mother's laughter, singing or speaking. Physical contact can also be used to comfort the infant. Such a treatment may be repeated regularly, for example weekly or monthly.

A second embodiment of the invention utilises the fact that from about 3-6 months in age the visual capabilities of the infant have developed sufficiently for them to recognise facial expressions, and in particular the mother's face. Thus in this embodiment the controlled exposure to fear pheromone is accompanied by a visual fear stimulus, such as a human facial expression of fear either from a human or in pictorial form. The use of the visual stimulus means that the correct fear pathways in the brain are associated both with the presence of the fear pheromone and fearful human expression. This therefore strengthens the correct synaptic connectivity related to the emotion of fear. Again the treatment may be repeated weakly or monthly.

Preferably the treatment can cease at six months of age as it has been found that the precipitating event for later development of schizophrenia tends to occur between 3 and 6 months of age.

With the invention the fear pathway in the brain is correctly exercised and established and does not latch onto incorrect stimuli such as human expressions of anger or disgust.

The invention is particularly effective if the infant can be placed in the psychological frozen state associated naturally with fear, this state then being relieved by the comforting stimuli following removal of the fear pheromone.

The invention also provides a human fear pheromone as described herein for use in a method of treatment of the human body by therapy.

The invention also provides a human fear pheromone as described herein for use in a method of improving the emotional development of an infant.

The invention also provides use of a human fear pheromone as described herein, in the manufacture of a medicament for use in a method of improving the emotional development of an infant.

FIG. 1 is a graph illustrating the development of the human visual cortex.

A study relating to the invention is described in the following non-limiting Example.

### Example 1

A study is carried out on a sample population of infants from three to six months old. All infants are isolated from their mothers and placed under the supervision of a clinical child psychologist. A liquid composition comprising human fear pheromone is sprayed in the vicinity of half the infants, who are simultaneously shown a picture of a human adult male having a fearful facial expression. Water is sprayed in the vicinity of the other infants, who are simultaneously shown a picture of a human adult male having a neutral facial expression. After five minutes of the foregoing procedure all infants are reintroduced to their mothers, and comforted by physical contact with their mothers and their mother's laughter. The procedure is repeated once a week for six weeks, maintaining the same groups of infants each time the procedure is repeated.

Following the six week treatment period the infants are assessed by a child psychologist every six months. The emotional development of the group subjected to the human fear pheromone is compared to the control group.

1. A method for improving the emotional development of an infant comprising performing controlled exposure of the infant to a human fear pheromone under medical supervision while the infant is less than six months old.

2. A method according to claim 1 wherein the controlled exposure is performed while the infant is from three to six months old.

3. A method according to claim 2 wherein the controlled exposure is performed while simultaneously providing a visual fear stimulus to the infant.

4. A method according to claim 4 wherein the visual fear stimulus is a fearful human expression.

5. A method according to claim 1 wherein the controlled exposure is performed while simultaneously inducing a frozen psychological state.

6. A method according to claim 1 wherein the controlled exposure is performed while the infant is less than three months old.

7. A method according to claim 1 wherein upon terminating the controlled exposure the infant is comforted.

8. A method according to claim 7 wherein the comfort is by at least one of feeding, soothing physical contact, soothing audible stimulation.

9. A method according to claim 1 wherein the human fear pheromone is a natural pheromone.

10. A method according to claim 1 wherein the human fear pheromone is a synthetic pheromone.

11. A method according to claim 1 wherein the human fear pheromone is a compound of Formula (1): 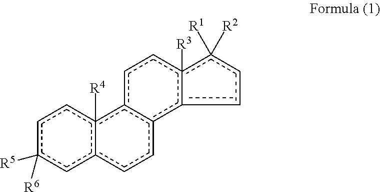
wherein:
R¹ and R² independently represent a hydrogen atom or a hydroxyl group, or R¹ and R² together represent an oxo group;
R³ represents a hydrogen atom or a methyl group, or is absent if the carbon atom to which it is attached is present in a carbon-carbon double bond
R⁵ and R⁶ independently represent a hydrogen atom or a hydroxyl group, or R⁵ and R⁶ together represent an oxo group;
R⁴ represents a hydrogen atom or a methyl group, or is absent if the carbon atom to which it is attached participates in a carbon-carbon double bond; and [symbol]
represents a carbon-carbon single bond or a carbon-carbon double bond, provided that no carbon atom participates in more than four bonds.

12. A method according to claim 1 in wherein the human fear pheromone is selected from compounds 1 to 7 below:

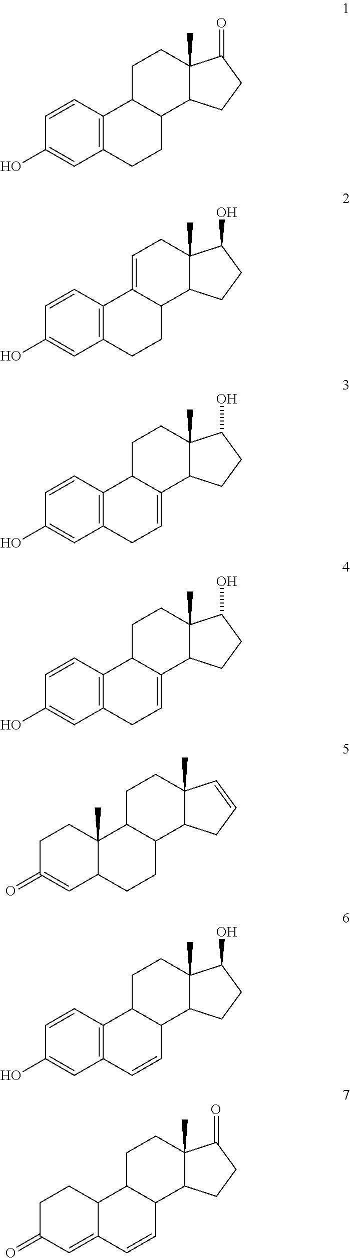

13. A method according to claim 1 wherein the human fear pheromone comprises cortisol.

14. A method according to claim 1 wherein the human fear pheromone is comprised in a fear-inducing composition.

15. A method according to claim 12 wherein the fear-inducing composition is sweat obtainable from a human subject in a state of fear.

16. A method according to claim 1 wherein the controlled exposure is repeated one of monthly and weekly.

17. A human fear pheromone for use in a method of improving the emotional development of an infant comprising performing controlled exposure of the infant to the human fear pheromone under medical supervision while the infant is less than six months old.

18. A human fear pheromone according for use according to claim 17 wherein the human fear pheromone is as defined in any one of claims 9-15.
